(19) United States
(12) Patent Application Publication
(10) Pub. No.: US 2003/0060880 A1
(43) Pub. Date: Mar. 27, 2003

(54) TORIC INTRAOCULAR LENS

(76) Inventor: VLADIMIR FEINGOLD, LAGUNA NIGUEL, CA (US)

Correspondence Address: KLIMA AND PEZZLO, P.O. BOX 2855, STAFFORD, VA, 22554

(21) Appl. No.: 08/790,100

(22) Filed: Jan. 29, 1997

Related U.S. Application Data
(63) Continuation of application No. 08/225,060, filed on Apr. 8, 1994, now abandoned.

(51) Int. Cl. A61F 2/16

(52) U.S. Cl. 623/6.27

(57) ABSTRACT

A toric shaped intraocular lens having at least one toric shape lens portion. The toric shaped intraocular lens corrects astigmatism of the eye. Preferably, the toric shaped intraocular lens is a deformable type intraocular lens.

### FIELD OF THE INVENTION

The present invention is directed to a toric intraocular lens. Specifically, the present invention is directed to an intraocular lens having a toric shaped lens portion on one or both sides of the lens.

### BACKGROUND OF THE INVENTION

A conventional intraocular lens is defined by a pair of spherical lens portions located on opposite sides of a centerplane of the lens. Typically, haptics or means of attachment of the lens to the eye are located in the centerplane.

In some conventional intraocular lens, a center portion of the lens can be defined by a planar piece of lens material having a circular center portion with a set of haptics extending from opposite sides thereof. The haptics can be defined by uniform thickness protrusions each having a hole therethrough for attachment in the eye by the capsule walls sewing to each through the hole. The spherical lens portions extend outwardly from opposite sides of the center portion.

The conventional intraocular lens made with spherical lens portions is not suitable for attending to astigmatism of the eye (i.e. a conventional lens is usually made with spherical surfaces that can only correct for spherical corneas). If astigmatism exist on the cornea, the conventional lens is unable to correct for the cylinder on the cornea. By making one or two surfaces toric shaped, these abnormalities are corrected resulting in perfect focus of the rays of light on the retina.

### SUMMARY OF THE INVENTION

An object of the present invention is to provide an improved intraocular lens.

Another object of the present invention is to provide an intraocular lens configured to correct astigmatism of the eye.

A further object of the present invention is to provide an intraocular lens having a toric shaped lens portion on one side of the lens.

An even further object of the present invention is to provide an intraocular lens having toric shaped lens portions on both sides of the lens.

Another object of the present invention is to provide an intraocular lens having a toric lens portion with a set of haptics extending from opposite sides thereof, and the haptics defined by uniform thickness or gradually variable thickness protrusions each having a hole therethrough for attachment in the eye by sutures.

The present invention is directed to a new type of intraocular lens having at least one toric-shaped lens portion, and to be referred to as a “toric intraocular lens (TIOL) hereinafter. Specifically, an intraocular lens can be made according to the present invention a first lens portion superimposed with respect to a second lens portion with the lens portions extending from opposite directions from a centerplane of the lens. In the present invention one or both of these lens portions can be toric-shaped. Other embodiments include one toric-shaped lens portion and one spherical-shaped lens portion. In addition, other embodiments can include one toric-shaped lens portion and one curved lens portion that is neither toric or spherical but some other type of mathematical shape (i.e. planar, convex, concave and other geometric shaped).

The “toric intraocular lens” according to the present invention includes an attachment for connecting the toric intraocular lens in the eye. For example, a pair of haptics can be provided for connecting the lens to the eye. Specifically, a pair of haptics extending from opposite sides of the lens portions and defined by substantially uniform thickness or gradually variable thickness protrusions having holes for connecting the lens by sutures in the eye. Other conventional haptics can be utilized with the lens according to the present invention for attachment in the eye.

In a preferred embodiment, the toric intraocular lens is provided with an alignment marker for aligning the lens in the eye during implantation. For example, a pair of linear protrusions located on opposite sides of at least one of the lens portions along a longitudinal axis of the lens can be provided for accurate placement in the eye. The protrusions are small extensions of the lens material that are formed during the molding of process and are outside the field of vision of the lens portion, however, can be seen by a physician during the implantation process.

The preferred embodiment of the toric intraocular lens according to the present invention is a one-piece molded lens wherein the lens portions and attaching haptics are molded out of the same material. Thus, the first lens portion is continuous with the second lens portion. Further, the lens portions are superimposed or centered back-to-back and extending from the centerplane of the lens.

The shape of the base of the toric-shaped lens portion can be circular, oval or some other suitable shape that provides a toric type shape of the lens (i.e. toric-shaped outer surface).

### BRIEF DESCRIPTION OF THE DRAWINGS

FIG. 1 is a perspective view of a preferred embodiment of the toric intraocular lens according to the present invention.

FIG. 2 is a top planar view of the toric intraocular lens shown in FIG. 1.

FIG. 3, is a cross-sectional view indicated as 3-3 in FIG. 2.

FIG. 4 is a cross-sectional view indicated as 4-4 in FIG. 2.

FIG. 5 is a blown up partial cross-sectional view along a longitudinal axis of the lens showing the alignment marks for positioning the lens in the eye.

FIG. 6 is a cross-sectional view as indicated as 6-6 in FIG. 7, of another embodiment of the toric intraocular lens according to the present invention.

FIG. 7 is a top planar view of the embodiment of the toric intraocular lens shown in FIG. 6.

### DETAILED DESCRIPTION OF PREFERRED EMBODIMENTS

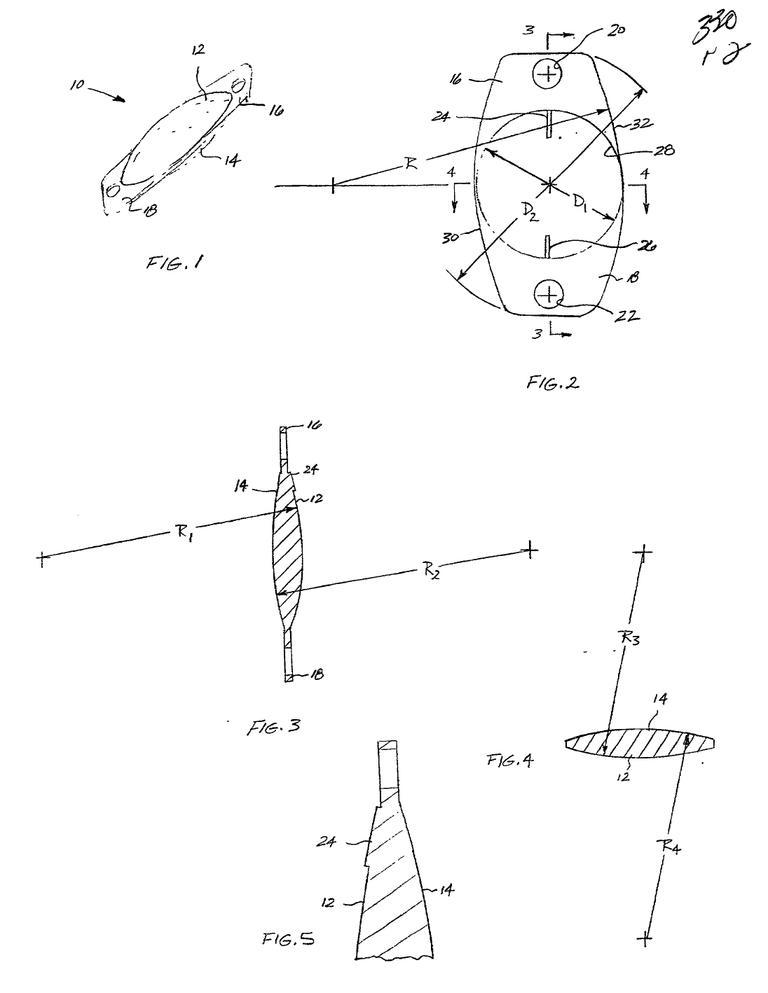

A perspective view of a toric intraocular lens 10 according to the present invention as shown in FIG. 1. The toric intraocular lens comprises a lens portion 12 located above a centerplane of the lens and a lens portion 14 located below the centerplane of the lens.

At least one of the lens portions 12 and/or 14 are toric-shaped. Specifically, one of the lens portions is toric-shaped and the other lens portion is toric-shaped, spherical, or some other suitable shape allowing a variety of combinations of toric intraocular lenses.

Referring to FIG. 2, toric intraocular lens 10 is provided with a pair of haptics 16 and 18. The haptic 16, 18 extend from opposite sides of the lens portions 12, 14. The haptics 16, 18 are provided with through holes 20, 22, respectively, for allowing the toric intraocular lens 10 to be affixed in the eye inside the capsular bag.

The haptic 16, 18 are defined by substantially uniform or variable thickness protrusions extending from opposite sides of the lens portions 12, 14. Other conventional type haptics can be substituted for the haptics 16, 18 shown and provide suitable means for affixing in the eye.

The toric intraocular lens 10 according to the present invention is preferably made by a molding process. Thus, the lens portions 12, 14 and haptics 16, 18 are made from the same material and are continuous forming a uniform one-piece structure.

The toric intraocular lens according to the present invention is preferable a formable intraocular lens that can be insert by a lens insertion system using a folded lens configuration to minimize the size of an incision in the eye. Suitable materials for the lens according to the present invention include flexible polymers such as silicone, acrylic, H.E.M.A., or any appropriate flexible material. The lens is preferably configured and made of a flexible polymer that will allow the lens to be inserted through an incision less than 3.5 mm into the eye.

In a preferred embodiment, the toric intraocular lens is provided with an alignment marker for use in aligning the lens within the eye during the implantation process. For example, a pair of linear protrusions 24, 26 can be molded at opposite sides of an outer peripheral portion of the lens portion 12, as shown in FIG. 2. The linear protrusions 24, 26 are located outside the field of vision, however, can be seen by a physician through the eye during the implantation for accurate alignment in the eye.

In the embodiment shown in FIG. 2, at least one of the lens portions 12, 14 is toric-shaped. In the embodiment shown, the lens portion 12 is toric-shaped with a circular base 28 having a diameter of D₁. The overall diameter of toric intraocular lens 10 is indicated as D₂. In addition, the sides 30, 32 are provided with a uniform radius indicated as R.

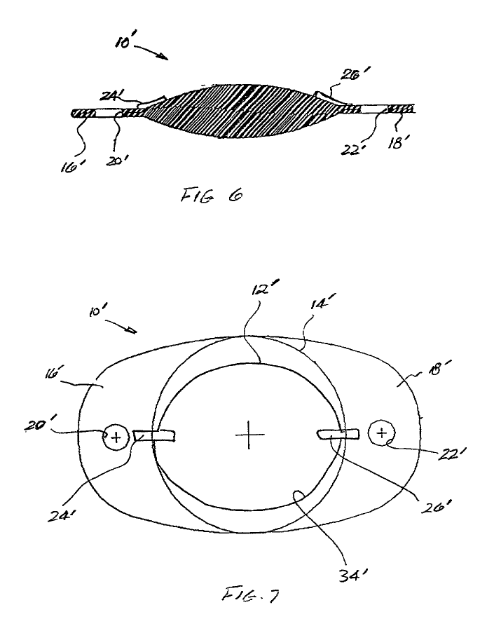

A cross-sectional shape along the axis 3-3 as shown in FIG. 3. The outer surface of the lens portion 12 as a radius of R₁. The outer surface of the lens portion 14 has a radius of R₂. A cross-sectional view of the lens as indicated as 4-4 in FIG. 2 is shown in FIG. 4. Along the axis perpendicular to the longitudinal axis of the lens, the lens portion 12 as an outer surface of radius R₃. Further, the lens portion 14 has a surface with an outer radius R₄.

In an embodiment where the lens portion 12 is toric, R₃ is greater than R₁. Further, the toric-shaped lens portion is preferably oriented in the centerplane of the lens so that the cross-section with the greatest curvature is perpendicular to the longitudinal axis of the lens, and the cross-section of least curvature is parallel to the longitudinal axis of the lens. In an embodiment where the lens portion 14 is spherical R₂ is equal to R₄. In an embodiment where the lens portion 14 is also toric, R₄ is greater than R₂. Other various combinations of curvature can be provided to correct various astigmatism. For example, the axis of greatest curvature and least curvature need not be located perpendicular to each other and these axis can be rotated to some degree of angle with respect to the longitudinal axis of the lens. The main concept is to provide at least one toric-shaped lens portion on at least one side of the toric intraocular lens.

Another embodiment of the toric intraocular lens according to the present invention is shown in FIGS. 6 and 7. In this embodiment, the toric-shaped lens portion 12′ has an oval-shaped base 34′ as shown. The lens portion 14′ can be toric-shaped of spherical shaped in two different embodiments.

is claimed is:

1. A toric intraocular lens for implantation into the eye, comprising:
a first toric shaped lens portion;
a second lens portion;
an attachment for connecting the lens to the eye.

2. A toric intraocular lens according to claim 1, wherein said second lens portion is toric shaped.

3. A toric intraocular lens according to claim 1, wherein said first toric shaped lens portion is oriented with an axis of least curvature positioned substantially parallel with a longitudinal axis of the lens.

4. A toric intraocular lens according to claim 1, wherein said first toric shaped lens portion is oriented with an axis of greatest curvature positioned perpendicular with a longitudinal axis of the lens.

5. A toric intraocular lens according to claim 3, wherein said first toric shaped lens portion is oriented with an axis of greatest curvature positioned perpendicular with a longitudinal axis of the lens.

6. A toric intraocular lens according to claim 1, wherein said lens portions are superimposed and extend outwardly in opposite directions from a centerplane of the lens.

7. A toric intraocular lens according to claim 1, wherein said attachment is defined by a pair of haptics.

8. A toric intraocular according to claim 7, wherein said haptics are defined by protrusions of substantially uniform thickness extending from opposite sides of said lens portions with each protrusions having a through hole to allow affixing the lens to the eye by sutures.

9. A toric intraocular lens according to claim 7, wherein said haptics are defined by protrusions having gradually variable thickness extending from opposite sides of said lens portions with each protrusion having a through hole to allow affixing the lens to the eye by sutures.

10. A toric intraocular lens according to claim 7, wherein said lens is provide with one or more alignment markers for facilitating alignment of the lens with respect to the eye during implantation.

11. A toric intraocular lens according to claim 10, wherein said alignment marker is defined by a pair of linear protrusions positioned at opposite ends of one of said lens portions and aligned with the longitudinal axis of the lens.

12. A toric intraocular lens according to claim 1, wherein the intraocular lens is a deformable intraocular lens made from a flexible polymer.

13. A toric intraocular lens according to claim 12, wherein said deformable intraocular lens is made of a flexible polymer of a type so that said deformable intraocular lens can be inserted through an incision less than 3.5 mm wide into the eye.

14. A toric intraocular lens according to claim 12, wherein said deformable intraocular lens is made of a flexible polymer material selected from the group consisting of silicone, acrylic, and H.E.M.A.
